(19) United States
(12) Patent Application Publication
(10) Pub. No.: US 2006/0160785 A1
(43) Pub. Date: Jul. 20, 2006

(54) Ezetimibe polymorphs

(76) Inventors: Judith Aronhime, Rehovot (IL); Tamas Koltai, Natania (IL); Guy Samburski, Ganot-Hadar (IL); Ori Lerman, Givatayim (IL); Reuven Izsak, Mizrah Binyamin (IL)

Correspondence Address: KENYON & KENYON LLP, ONE BROADWAY, NEW YORK, NY, 10004, US

(21) Appl. No.: 11/295,141

(22) Filed: Dec. 5, 2005

Related U.S. Application Data
(60) Provisional application No. 60/632,543, filed on Dec. 3, 2004.
(60) Provisional application No. 60/649,139, filed on Feb. 3, 2005.
(60) Provisional application No. 60/668,571, filed on Apr. 6, 2005.
(60) Provisional application No. 60/687,316, filed on Jun. 6, 2005.
(60) Provisional application No. 60/712,781, filed on Aug. 30, 2005.
(60) Provisional application No. 60/717,275, filed on Sep. 14, 2005.

(51) Int. Cl. A61K 31/397; C07D 205/02

(52) U.S. Cl. 514/210.02; 540/200

(57) ABSTRACT

Provided are processes for preparing crystalline forms of ezetimibe, such as ezetimibe Form A or Form B, for example, by precipitating ezetimibe from selected solvents. Alternatively, some forms may be transformed into different forms at elevated temperatures or under various humidity conditions, or by micronization. Also provided are micronized ezetimibe Form A, micronized ezetimibe Form B, and ezetimibe having a plate morphology. Pharmaceutical compositions containing these forms are particularly useful in reducing cholesterol in patients in need thereof.

The present application claims the benefit of the following U.S. Provisional Patent Applications Nos.: 60/632,543 filed Dec. 3, 2004, 60/649,139 filed Feb. 3, 2005, 60/668,571 filed Apr. 6, 2005, 60/687,316 filed Jun. 6, 2005, 60/712,781 filed Aug. 30, 2005 and 60/717,275 filed Sep. 14, 2005. The contents of which are incorporated herein by reference.

### FIELD OF THE INVENTION

The present invention relates to micronized crystalline forms of ezetimibe and methods of preparing crystalline and amorphous forms of ezetimibe.

### BACKGROUND OF THE INVENTION

Ezetimibe, or 1-(4-fluorophenyl)-3(R)-[3-(4-fluorophenyl)-3(S)-hydroxypropyl]-4(S)-(4-hydroxyphenyl)-2-azetidinone, has the following chemical structure:

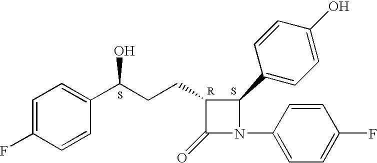

It is a white crystalline powder that is freely to very soluble in ethanol, methanol, and acetone, and practically insoluble in water. Ezetimibe is reported to have a melting point of about 163° C. and to be stable at ambient temperature.

Ezetimibe is in a class of lipid-lowering compounds that selectively inhibits the intestinal absorption of cholesterol and related phytosterols. It is reported that ezetimibe has a mechanism of action that differs from those of other classes of cholesterol-reducing compounds, such as HMG-CoA reductase inhibitors, bile acid sequestrants (resins), fibric acid derivatives, and plant stanols. Ezetimibe reportedly does not inhibit cholesterol synthesis in the liver or increase bile acid excretion. Instead, it appears that ezetimibe localizes and acts at the brush border of the small intestine and inhibits the absorption of cholesterol, leading to a decrease in the delivery of intestinal cholesterol to the liver. The result is a reduction of hepatic cholesterol stores and an increase in clearance of cholesterol from the blood. Such a mechanism is complementary to that of HMG-CoA reductase inhibitors.

Ezetimibe is sold under the brand name Zetia®, which is marketed by Merck/Schering-Plough Pharmaceuticals. Zetia® is available as a tablet for oral administration containing 10 mg of ezetimibe and the following inactive ingredients: croscarmellose sodium NF, lactose monohydrate NF, magnesium stearate NF, microcrystalline cellulose NF, povidone USP, and sodium lauryl sulfate NF.

WO patent No. 2005/009955 discloses two crystalline forms of ezetimibe and processes for preparation thereof. The first form may be characterized by XRD peaks at about 20.2, 22.5, 23.1, 23.7, 23.9, 25.7, 28.1 and 29.8 degrees two-theta, +0.2 degrees two-theta, and the second form may be characterized by XRD peaks at about 16.4, 18.6, 19, 19.4, 20.2, 22.4, 22.9, 23.6, 23.9, 25.6, 27.9 and 29.7 degrees two-theta, ±0.2 degrees two-theta.

Polymorphism, the occurrence of different crystal forms, is a property of some molecules and molecular complexes. A single molecule, like ezetimibe, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

J. Org. Chem. 1999, 64, 3717 discloses the preparation of crystalline ezetimibe by crystallization from a mixture of methyltertbutyl ether and heptane, followed by a second crystallization from a mixture of methanol and water.

U.S. Pat. No. 5,886,171 discloses the preparation of crystalline ezetimibe by crystallization from an aqueous solution of iso-propanol.

U.S. Pat. No. 6,207,822, discloses the preparation of crystalline ezetimibe by crystallization from a mixture of methyltertbutyl ether and heptane, followed by a second crystallization from a mixture of methanol and water.

When the crystallization processes disclosed in U.S. Pat. No. 6,207,822 and in U.S. Pat. No. 5,886,171 are repeated, crystalline ezetimibe having needle-shape morphology is obtained, as substantially depicted in FIGS. 15a and 15b.

There is a need in the art for processes to obtain crystalline forms of ezetimibe.

### SUMMARY OF THE INVENTION

In one aspect, the present invention encompasses a process for obtaining ezetimibe Form B comprising combining ezetimibe with a solvent including at least one solvent selected from the group consisting of methyl isobutyl ketone, dichloromethane, chloroform, and ethylacetate to obtain a mixture; heating the mixture at a temperature sufficient to obtain a solution; precipitating the ezetimibe from the solution; and optionally recovering the precipitate.

In another aspect, the present invention encompasses a process for obtaining a mixture of ezetimibe Form A and Form B comprising combining ezetimibe with a solvent including at least one solvent selected from the group consisting of n-butanol, n-propanol, butylacetate, bromobenzene, chlorobenzene, dibromomethane, xylene, toluene, acetonitrile, nitromethane, and isobutanol to obtain a mixture; heating the mixture at a temperature sufficient to obtain a solution; precipitating the ezetimibe from the solution; and optionally recovering the precipitate.

In yet another aspect, the present invention encompasses a process for obtaining ezetimibe Form A comprising combining ezetimibe with a solvent including isoamyl alcohol to obtain a mixture; heating the mixture at a temperature sufficient to obtain a solution; precipitating the ezetimibe from the solution; and optionally recovering the precipitate.

In one aspect, the present invention encompasses a process for obtaining amorphous ezetimibe comprising combining ezetimibe with a solvent including at least one solvent selected from the group consisting of ethylene glycol and 2-butanol to obtain a mixture; heating the mixture at a temperature sufficient to obtain a solution; precipitating the ezetimibe from the solution; and optionally recovering the precipitate.

In another aspect, the present invention encompasses a process for obtaining ezetimibe Form B comprising: combining ezetimibe with a solvent including at least one solvent selected from the group consisting of an ether, a ketone, an amide, methanol, ethanol, 2-propanol, and propylene glycol to obtain a mixture; heating the at a temperature sufficient to obtain a solution; combining the solution with a solvent including at least one anti-solvent selected from the group consisting of water and a cyclic or linear C₅₋₆ aliphatic hydrocarbon to obtain a suspension; precipitating the ezetimibe from the suspension; and optionally recovering the precipitate.

In yet another aspect, the present invention encompasses a process for obtaining amorphous ezetimibe comprising combining ezetimibe with a solvent including propylene glycol to obtain a mixture; heating the mixture at a temperature sufficient to obtain a solution; combining the solution with a solvent including at least one anti-solvent selected from the group consisting of water and a cyclic or linear C₅₋₆ aliphatic hydrocarbon to obtain a suspension; precipitating the ezetimibe from the suspension; and optionally recovering the precipitate.

In one aspect, the present invention encompasses a process for preparing ezetimibe Form B comprising slurrying ezetimibe Form A in a solvent including at least one solvent selected from the group consisting of water and a C₁₋₄ alcohol.

In another aspect, the present invention encompasses a process for preparing ezetimibe Form B comprising combining ezetimibe with a C₁₋₄ alcohol to obtain a solution; combining the solution with water to obtain a precipitate; recovering the precipitate; and recrystallizing the precipitate.

In yet another aspect, the present invention encompasses a process for preparing ezetimibe Form A comprising maintaining ezetimibe Form B or amorphous ezetimibe at a temperature of about 40° C. to about 110° C. for about 2 hours to about 18 hours.

In one aspect, the present invention encompasses a process for preparing ezetimibe Form B comprising exposing ezetimibe Form A to a relative humidity of about 40% to about 100% for about 1 day to about 14 days at a temperature of about 25° C. to about 30° C.

In another aspect, the present invention encompasses a process for preparing ezetimibe Form A comprising exposing ezetimibe Form B to a relative humidity of about 0% to about 20% for about 7 days to about 14 days at a temperature of about 25° C. to about 30° C.

In yet another aspect, the present invention encompasses a process for preparing Form A comprising micronizing Form B.

In another aspect, the present invention encompasses a process for preparing Form B by exposing a mixture of micronized Form A and micronized Form B to a relative humidity of about 40% to about 100% at a temperature of 25° C. about 30° C. for about 7 to about 14 days.

In one aspect, the present invention encompasses micronized ezetimibe Form A. In another aspect, the present invention encompasses micronized ezetimibe Form B. In yet another aspect, the present invention encompasses ezetimibe having a plate morphology.

In one aspect, the present invention encompasses a pharmaceutical composition comprising the ezetimibe of the present invention, and at least one pharmaceutically acceptable excipient.

In another aspect, the present invention encompasses a process for preparing a stable pharmaceutical formulation comprising combining the ezetimibe made of the present invention with at least one pharmaceutically acceptable excipient

### BRIEF DESCRIPTION OF THE FIGURES

FIG. 1 illustrates the powder X-ray diffraction pattern for ezetimibe Form A.

FIG. 2 illustrates the powder X-ray diffraction pattern for ezetimibe Form B.

FIG. 3 illustrates the powder X-ray diffraction pattern for a mixture of 80% Form A and 20% Form B by weight.

FIG. 4 illustrates the powder X-ray diffraction pattern for a mixture of 50% Form A and 50% Form B by weight.

FIG. 5 illustrates the powder X-ray diffraction pattern for a mixture of 20% Form A and 80% Form B by weight.

FIG. 6 illustrates the powder X-ray diffraction pattern for a mixture of 10% Form A and 90% Form B by weight.

FIG. 7 illustrates Form B before micronization as seen through a microscope.

FIG. 8 illustrates Form B after micronization as seen through a microscope.

FIG. 9(a) illustrates the powder X-ray diffraction pattern for the essentially amorphous form of ezetimibe.

FIGS. 9(b) and 9(c) illustrate the crystallinity of two samples of the essentially amorphous form of ezetimibe.

FIG. 10 illustrates the powder X-ray diffraction pattern for the purely amorphous form of ezetimibe.

FIG. 11 illustrates the PXRD diffractogram of Form A of ezetimibe before micronization.

FIG. 12 illustrates the PXRD diffractogram of Form A of ezetimibe after micronization.

FIG. 13 illustrates micronized Form A as seen through a microscope.

FIG. 14 illustrates micronized Form A as seen through a microscope.

FIGS. 15(a) and 15(b) illustrate wet ezetimibe having a needle-shaped morphology as seen through a microscope.

FIG. 16 illustrates ezetimibe having a plate-shaped morphology as seen through a microscope.

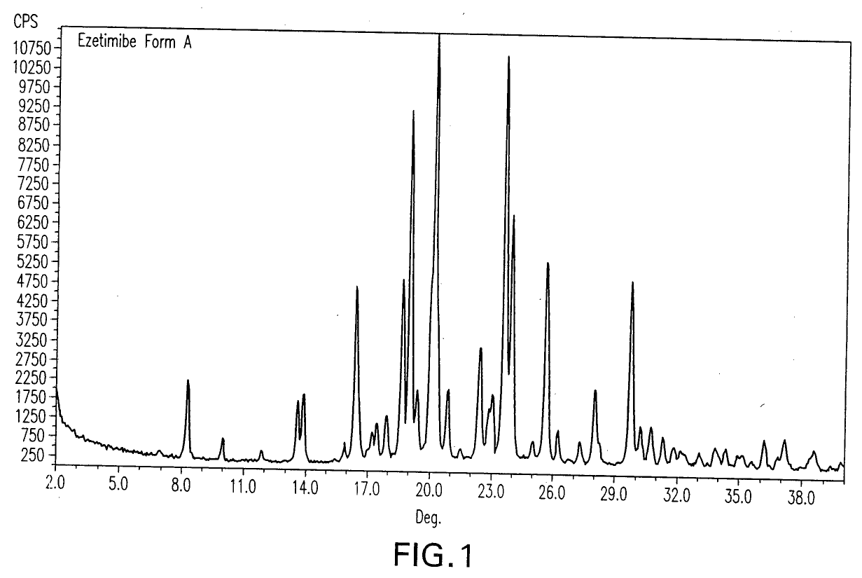

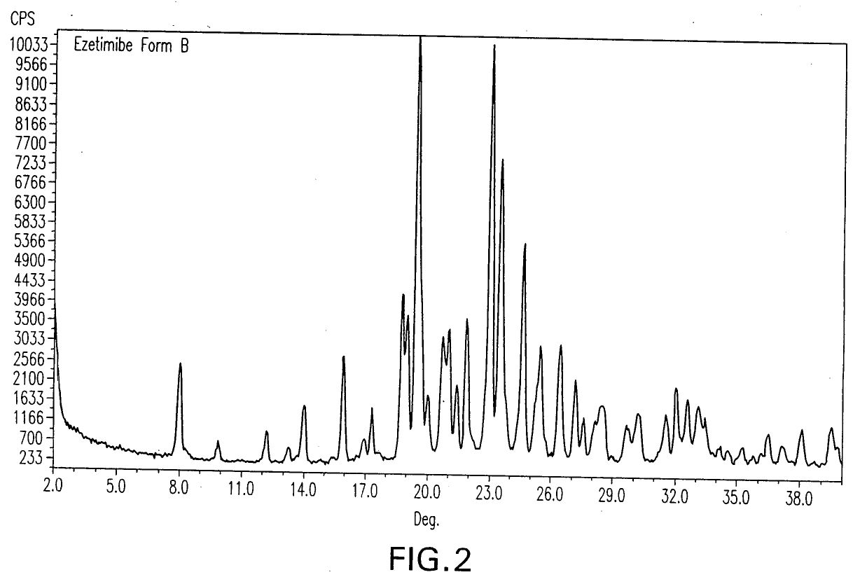

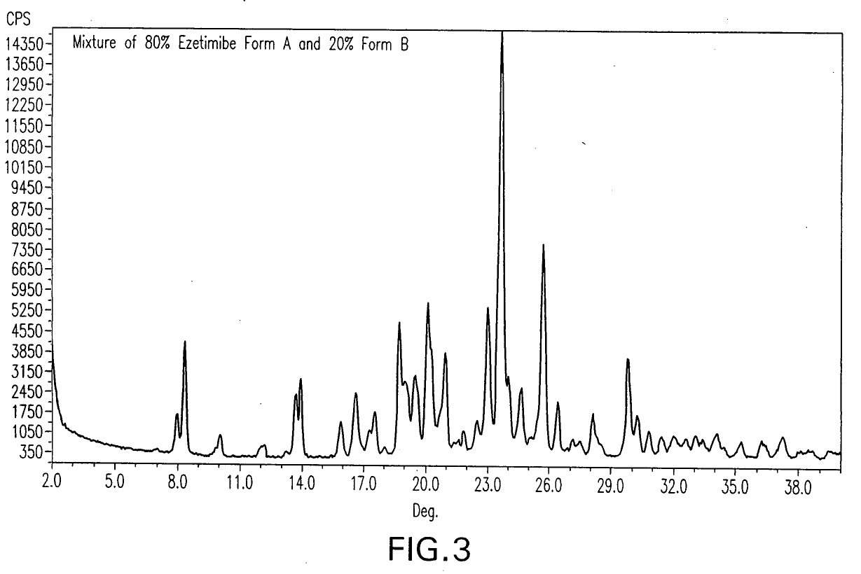

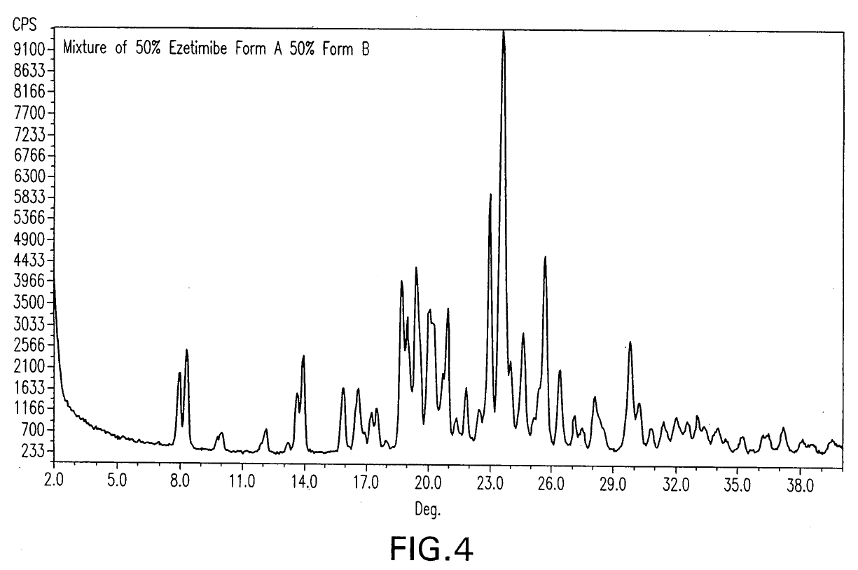

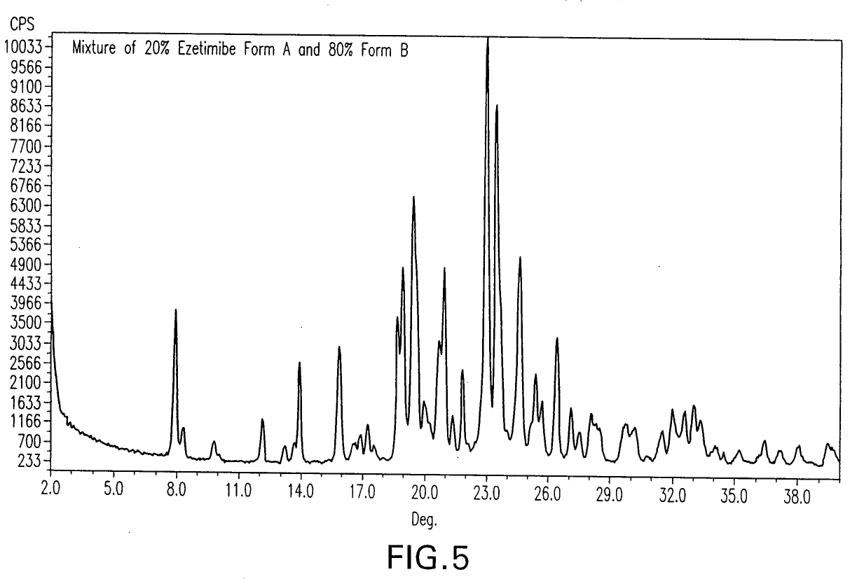

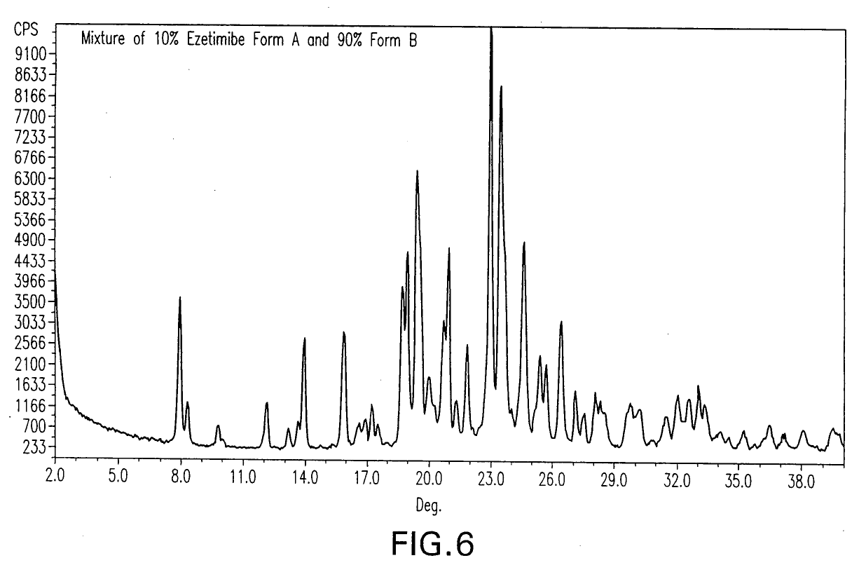

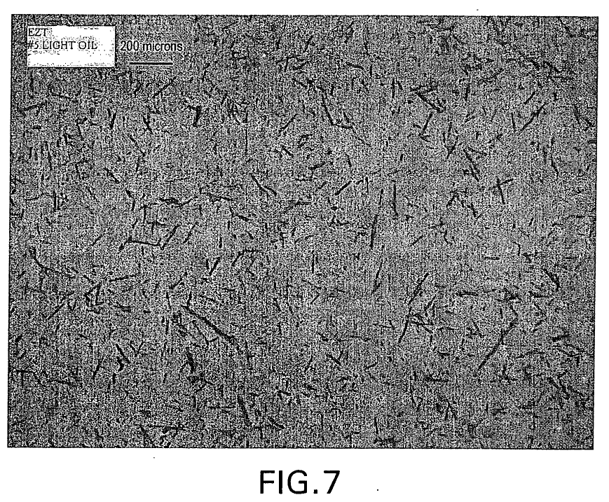

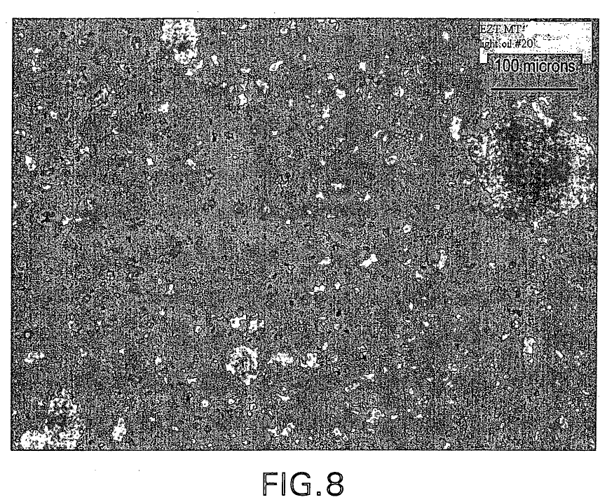

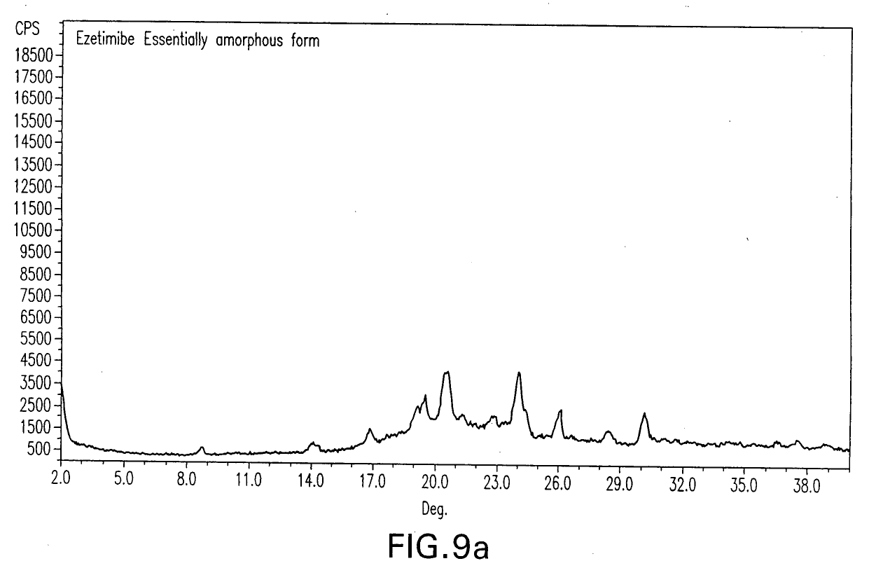

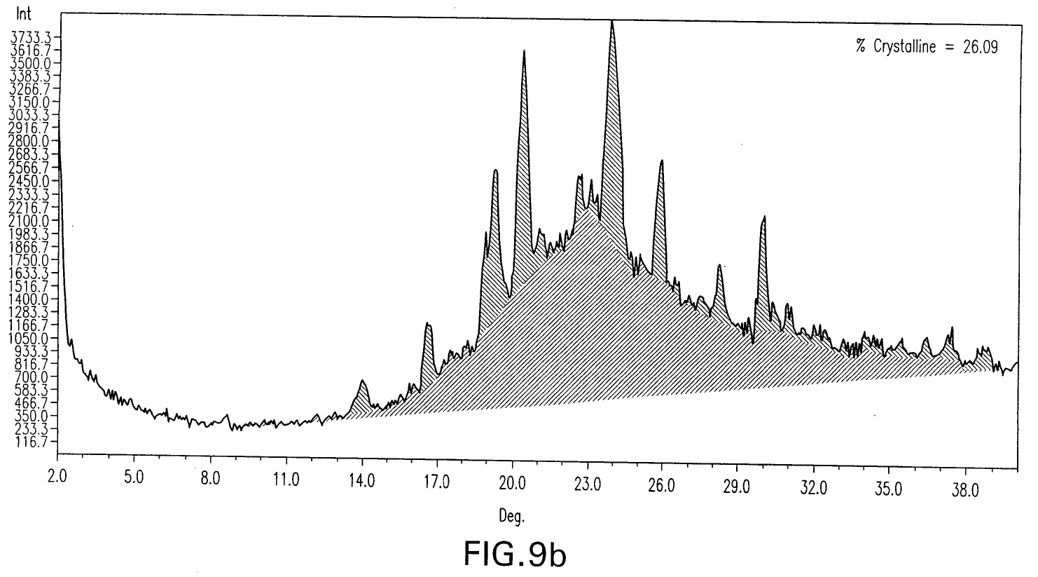

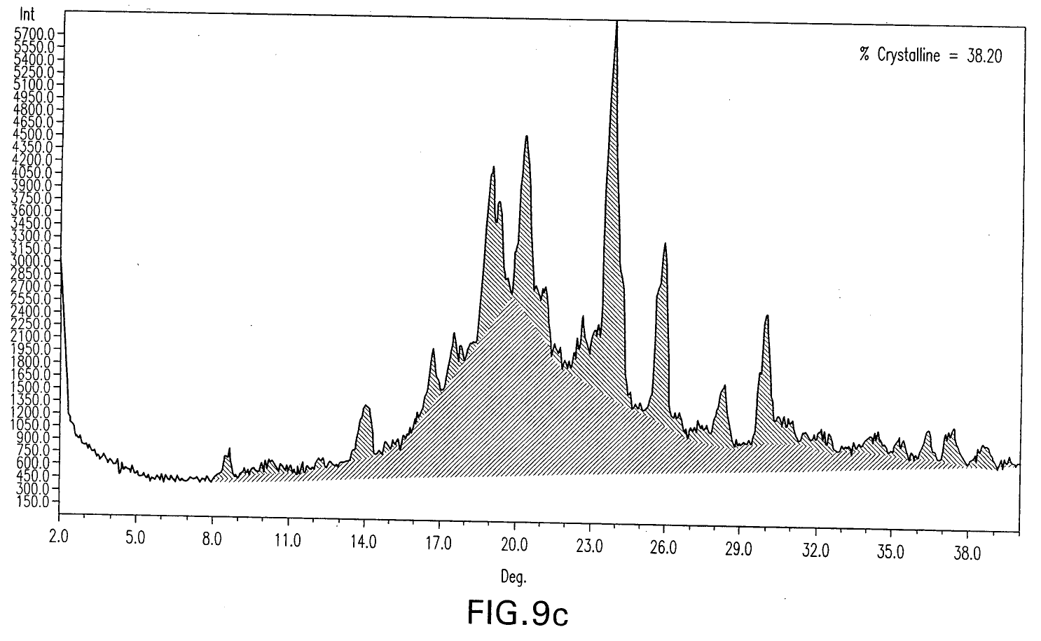

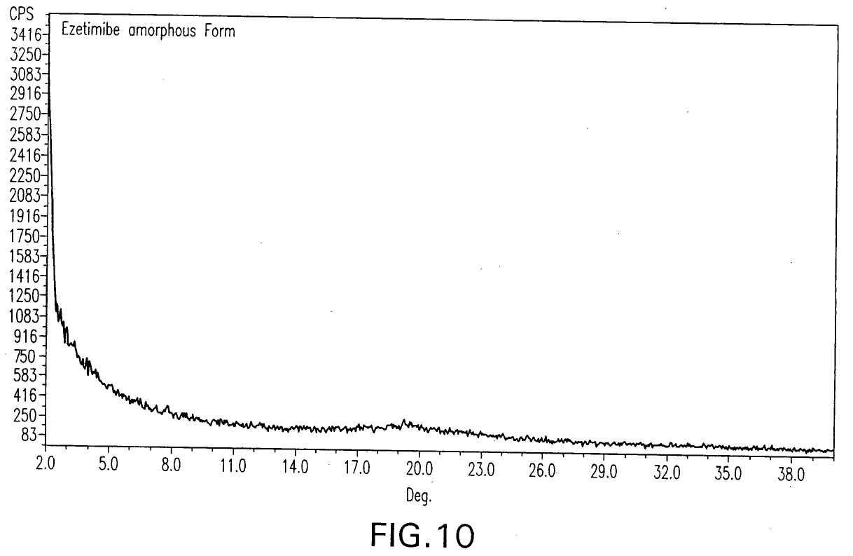

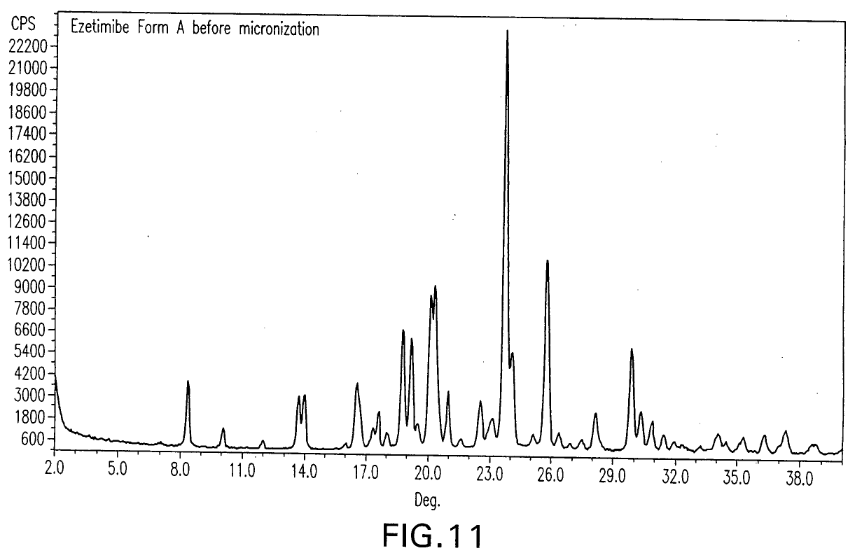

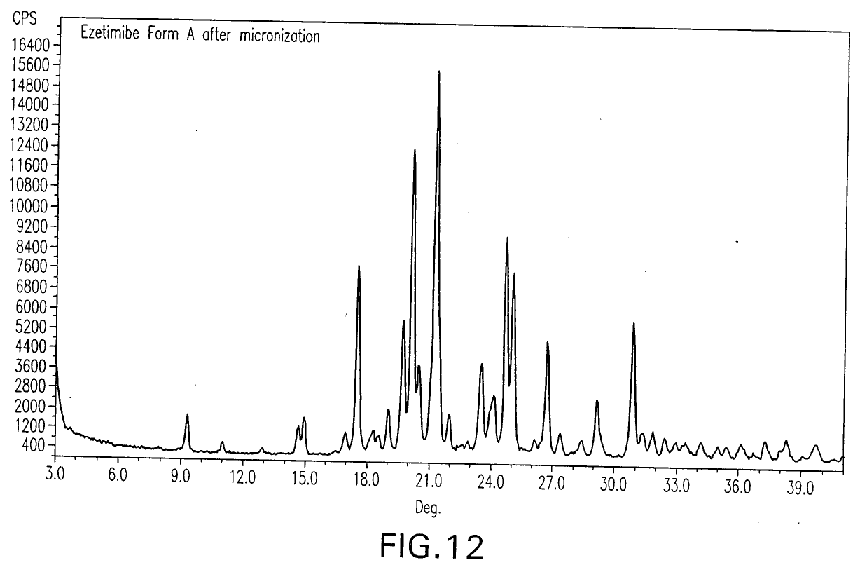

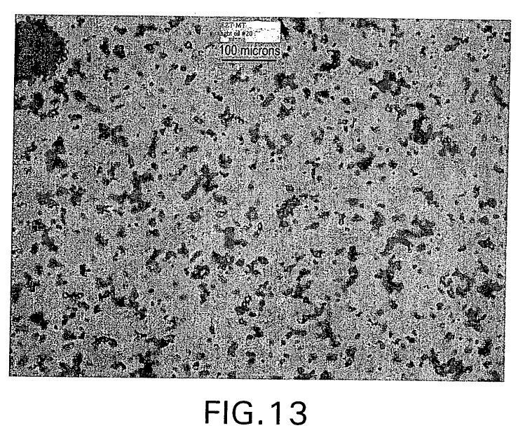

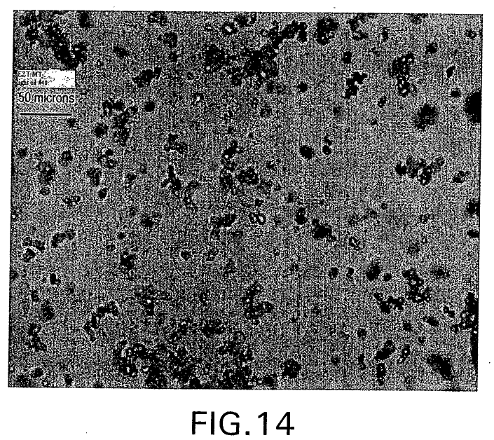

### DETAILED DESCRIPTION OF THE INVENTION

As used herein, the term “wet precipitate” refers to a precipitate containing more than 5% of solvent, as determined by XRD.

As used herein, the term “stable” refers to a polymorph that converts by about 5% or less, as determined by XRD, to other polymorphs.

Crystalline ezetimibe Form A is an anhydrous form characterized by powder X-ray diffraction peaks at 16.4, 20.2, 22.5, 24.0, and 25.6 degrees two-theta, +0.2 degrees two-theta and by additional peaks at about 8.2, 18.6, 19.0, 23.6, and 29.7 degrees two-theta, +0.2 degrees two-theta. Form A can be characterized by a water content of about 0.1% by weight, as determined by a weight loss measurement by thermal gravimetric analysis (TGA). Form A can also be characterized by a water content of about 0.2% by weight, as determined by Karl Fisher (KF) analysis.

Crystalline ezetimibe Form B is characterized by powder X-ray diffraction peaks at 18.7, 19.5, 23.0, 23.5 and 24.6 degrees two-theta, +0.2 degrees two-theta, and by additional peaks at about 8.0, 15.9, 20.7, 21.9 and 25.5 degrees two-theta, +0.2 degrees two-theta. Form B can be characterized by a water content of about 4% to about 28% by weight, as determined by TGA analysis. Form B can also be characterized by a water content of about 3% to about 23% by weight, as determined by Karl Fisher analysis. The present invention provides a process for obtaining Form B of ezetimibe by combining ezetimibe with a solvent including at least one solvent selected from the group consisting of methylisobutyl ketone, dichloromethane, chloroform and ethylacetate to obtain a mixture, which is then converted to a solution by heating at a temperature sufficient to obtain a solution, preferably a temperature of about 40° C. to about 200° C. The ezetimibe is then precipitated, preferably by cooling, more preferably cooled to a temperature of about room temperature to about 4° C., optionally followed by recovery of the obtained precipitate.

Preferably, the mixture is heated to a temperature of about 40° C. to about 140° C., and more preferably with stirring. Preferably, the solution is maintained at a temperature of about 25° C. to about 4° C., for about 4 to about 24 hours, to obtain a precipitate.

The precipitate of Form B may be a wet precipitate, which can be recovered by filtration, preferably, by using a Buchner funnel, followed by sucking air to dry the precipitate. The wet precipitate of Form B is preferably dried under reduced pressure for about 14 to about 24 hours, to obtain a dry precipitate of Form B.

The present invention further provides a process for obtaining a mixture of ezetimibe Form A and Form B by combining ezetimibe with a solvent including at least one solvent selected from the group consisting of n-butanol, n-propanol, butylacetate, bromobenzene, chlorobenzene, dibromomethane, dibromobutane, xylene, toluenene, acetonitrile, nitromethane and isobutanol to obtain a mixture, which is then converted to a solution by heating at a temperature sufficient to obtain a solution, preferably a temperature of about 40° C. to about 200° C. The ezetimibe is then precipitated, preferably by cooling, more preferably cooled to a temperature of about room temperature to about 4° C. to induce precipitation, optionally followed by recovery of the obtained precipitate.

Preferably, the mixture is heated to a temperature of about 80° C. to about 140° C., and more preferably with stirring. Preferably, the solution is maintained at a temperature of about 25° C. to about 4° C., for about 4 to about 24 hours, to obtain a precipitate.

The precipitate of the mixture of Form A and Form B may be a wet precipitate, which can recovered and dried to obtain a dry precipitate of Form A, in a similar fashion described in the crystallization process to obtain Form B.

Preferably, the mixture of Form A and Form B contains about 10% to about 99% of Form A or about 10% to about 99% of Form B, as determined by XRD or by KF analysis.

The present invention also provides a process for obtaining a ezetimibe Form A by combining ezetimibe with a solvent including isoamyl alcohol to obtain a mixture, which is then converted to a solution by heating at a temperature sufficient to obtain a solution, preferably a temperature of about 40° C. to about 200° C. The ezetimibe is then precipitated, preferably by cooling, more preferably cooled to a temperature of about room temperature to about 4° C. to induce precipitation, optionally followed by recovery of the obtained precipitate.

Preferably, the mixture is heated to a temperature of about 40° C. to about 120° C., and more preferably with stirring. Preferably, the solution is maintained at a temperature of about 25° C. to about 4° C., for about 4 to about 24 hours, to obtain a precipitate.

The precipitate of Form A may be a wet precipitate, which can be recovered and dried to obtain a dry precipitate of Form A, in a similar fashion described in the crystallization process to obtain Form B.

The present invention provides a process for obtaining amorphous form of ezetimibe by combining ezetimibe with a solvent including at least one solvent selected from the group consisting of ethylene glycol and 2-butanol to obtain a mixture, which is then converted to a solution by heating at a temperature sufficient to obtain a solution, preferably a temperature of about 40° C. to about 200° C. The ezetimibe is then precipitated, preferably by cooling, more preferably cooled to a temperature of about room temperature to about 4° C. to induce precipitation, optionally followed by recovery of the obtained precipitate.

Preferably, the mixture is heated to a temperature of about 90° C. to about 120° C., and more preferably with stirring. Preferably, the solution is maintained at a temperature of about 25° C. to about 4° C., for about 4 to about 24 hours, to obtain a precipitate.

The precipitate of the amorphous form may be a wet precipitate, which can be recovered and dried to obtain a dry precipitate, either of amorphous form or of Form A, in a similar fashion described in the crystallization process to obtain Form B.

The present invention further provides a process for obtaining ezetimibe Form B by combining a solvent including ezetimibe with at least one solvent selected from the group consisting of ether, ketone, amide, methanol, ethanol, 2-propanol, and propylene glycol to obtain a mixture, which is then converted to a solution by heating at a temperature sufficient to obtain a solution, preferably a temperature of about 34° C. to about 190° C. The solution is then combined with at least one second solvent, an anti-solvent, selected from the group consisting of water and a cyclic or linear C₅₋₆ aliphatic hydrocarbon to obtain a suspension. The ezetimibe is then precipitated, preferably by cooling, more preferably cooled to a temperature of about room temperature to about 4° C. to give a precipitate, optionally followed by its recovery.

Preferably, the ether is tetrahydrofuran, diethyletner, t-butyl-methylether, 1,3-dioxalane or 1,4-dioxane. A preferred ketone is either acetone or methylethlyl ketone. Preferably, the amide is N,N-dimethylformamide. Preferably, the cyclic or linear C₅₋₆ aliphatic hydrocarbon is cyclohexane.

Preferably, the mixture is heated to a temperature of about 40° C. to about 140° C.

Preferably, the suspension is maintained at a temperature of about 25° C. to about 4° C., for about 4 to about 24 hours, to obtain a precipitate.

The precipitate of Form B may be a wet precipitate, which can be recovered and dried, as described in the crystallization process to obtain Form A.

The present invention also provides a process for obtaining amorphous form of ezetimibe by combining ezetimibe with a solvent including propylene glycol to obtain a mixture, which is then converted to a solution by heating at a temperature sufficient to obtain a solution, preferably a temperature of about 34° C. to about 190° C. The solution is then combined with at least one second solvent, an anti-solvent, which is a cyclic or linear C₅₋₆ aliphatic hydrocarbon, to obtain a suspension. The ezetimibe is then precipitated, preferably by cooling, more preferably cooled to a temperature of about room temperature to about 4° C. to give a precipitate.

Preferably, the mixture is heated to a temperature of about 40° C. to about 140° C., and more preferably with stirring. Preferably, the suspension is maintained at a temperature of about 25° C. to about 4° C., for about 4 to about 24 hours, to obtain a precipitate.

The amorphous form may be a wet precipitate, which can be recovered and dried to obtain a dry precipitate of the amorphous form, in a similar fashion described in the crystallization process to obtain Form A.

The present invention provides a process for preparing ezetimibe Form B by slurrying ezetimibe Form A in a solvent including at least one solvent selected from the group consisting of water and C₁₄ alcohol.

Preferably, the C₁₄ alcohol is an absolute C ₁₄ alcohol, and more preferably, absolute ethanol or absolute methanol.

Preferably, slurrying is done with stirring. Preferably, slurrying is done at a temperature of about 15° C. to about 30° C. Preferably, the slurry is maintained at a temperature of about 15° C. to about 30° C. for about 3 to about 8 hours.

Drying the obtained Form B at a temperature of about 40° C. to about 60° C. under reduced pressure of about 16 to about 20 mm Hg for about 10 to about 24 hours, may lead to a mixture containing about 20% to about 30% of Form B and 80% to about 70% of form A, as determined by XRD or by Karl Fisher, or a complete transformation to Form A, depending on the solvent and its amount. Preferably, at least 99% of Form B is transformed to Form A, as determined by XRD or KF analysis.

The present invention further provides a process for preparing ezetimibe Form B by combining ezetimibe with a solvent including C₁₄ alcohol to obtain a solution, which is then combined with water to obtain a precipitate, followed by recovering and recrystallizing the precipitate.

Preferably, the C₁₄ alcohol is ethanol. The precipitate may be recovered by filtering, followed by drying.

The recovered precipitate may be further purified by recrystallization, such as by repeating the crystallization process for preparing Form B. The precipitate obtained may be a wet precipitate of Form B, which after drying gives Form B.

Preferably, the wet precipitate of Form B is dried in the hood for about 1 to about 4 days.

Form B obtained by the above recrystallization process preferably contains about 3% to about 5% water by weight, and more preferably about 4.1% of water by weight as determined by KF analysis.

Amorphous form may also be prepared by combining ezetimibe with a solvent including at least one organic solvent selected from the group consisting of a ketone, halogenated C₁₋₂ aliphatic hydrocarbon, an ether, and a C₁₋₂ alcohol to obtain a solution, followed by heating at a temperature sufficient to obtain a solution, preferably a temperature of about 30° C. to about 100° C., preferably under reduced pressure of about 50 to about 10 mm Hg.

Preferably, the solution is heated to a temperature of about 40° C. to about 80° C. Preferably, the ketone is acetone. A preferred halogenated C₁₋₂ aliphatic hydrocarbon is dichloromethane. A preferred ether is tetrahydrofuran, diethylether or methyltertbutyl ether. Preferably, the C₁₋₂ alcohol is methanol.

The present invention provides a process for preparing ezetimibe Form A by maintaining ezetimibe Form B or amorphous ezetimibe at a temperature of about 40° C. to about 110° C. for about 2 hours to about 18 hours.

Preferably, the transformation to Form A occurs in less than about 2 hours. One of ordinary skill in the art will appreciate that the transformation time may depend on the drying conditions.

Ezetimibe Form A is stable upon heating, even at high temperatures. Amorphous form of ezetimibe transforms to Form A at temperatures above 60° C.

The present invention further provides a process for preparing ezetimibe Form B by exposing ezetimibe Form A to a relative humidity of about 40% to about 100% for about 1 day to about 14 days at a temperature of about 25° C. to about 30° C.

Preferably, when the relative humidity is about 100%, transformation of From A to Form B occurs in about 1 day.

The present invention also provides a process for preparing ezetimibe Form A by exposing ezetimibe Form B to a relative humidity of about 0% to about 20% for about 7 days to about 14 days at a temperature of about 25° C. to about 30° C.

Preferably, transformation of From B to Form A occurs in less than about 3 days.

When the relative humidity is of less than about 20% the crystalline form obtained is Form A in an amount greater than any other single ezetimibe polymorphic form by weight, and preferably about 90% to about 95% by weight, as determined by XRD or by KF. When the relative humidity is of about 0%, about 100% of Form A by weight is obtained.

The present invention also provides a process for preparing Form A by micronization of Form B.

Micronization of Form B is preferably carried out by milling Form B using a feed air rate of about 6 bar and a grinding air pressure of about 5 bar, for about 20 to about 30 minutes.

When the milling is done for a maximum of about 30 minutes, the resultant Form A may contain about 35% of form B and about 1% to about 2% of water by weight, as determined by XRD or by KF. If milling is conducted for more than 30 minutes, complete transformation of Form B to Form A, as determined by XRD or by KF, may occur.

When Form A is milled under the same conditions, micronized Form A is obtained. Therefore, Form A is stable upon micronization.

The present invention further provides a process for preparing Form B by exposing a mixture of micronized Form A and micronized Form B to a relative humidity of about 40% to about 100% at a temperature of 25° C. about 30° C. to about for about 7 to about 14 days.

Preferably, the Form B obtained contains about 3% to about 5% of water by weight, and more preferably about 4.1% of water by weight as determined by XRD or by KF.

The mixture of micronized Form A and micronized Form B may be obtained, for example, by the micronization process of the present invention.

Preferably, the transformation of the mixture of micronized Form A and micronized Form B to Form B occurs in less than 7 days.

The present invention also provides micronized ezetimibe Form A. Ezetimibe Form A is stable upon micronization.

The present invention also provides micronized ezetimibe Form B. Micronized Form B is stable in relative humidity of about 40% to about 100% and upon slurrying.

Ezetimibe is practically insoluble in water (0.01 mg/ml). Micronized ezetimibe has significant pharmaceutical advantages. For example, micronized ezetimibe has a much higher specific surface area (SSA) than the non-micronized form. An increase in the SSA of low aqueous solubility materials may improve therapeutic activity.

Micronized ezetimibe forms may be characterized by particle size or by specific surface area. Preferably, at least about 99% of micronized ezetimibe particles have a particle size of less than about 30 microns, more preferably less than about 20 microns, and most preferably less than about 10 microns.

The size of a particle is determined by any of the methods commonly known in the art. The following methods, for example, may be used: sieves, sedimentation, electrozone sensing (coulter counter), microscopy, or Low Angle Laser Light Scattering (LALLS). The preferred methods for the present invention are the methods most commonly used in the pharmaceutical industry, such as laser diffraction, sieve analysis or microscope observation. Particle size is preferably determined by microscope observation.

Micronized ezetimibe has a specific surface area (SSA) of about 5 m 2/g to about 8 m²/g, preferably, of about 6 m²/g to about 7 m²/g, and more preferably about 6.8 m²/g. Specific surface area may be measured by any method accepted in the pharmaceutical industry with the provision that the result obtained is reasonably accurate, i.e., within widely accepted industrial standards.

Crystalline stability of Form A and Form B was tested by pressing each of the forms for about 1 minute under a pressure of about 1300 psi. Form B retains its crystalline form under the testing conditions. Form A is also stable under pressing.

Quantification of the amount of Form B in a mixture of ezetimibe Form A and Form B is done by determining the water content of the mixture by KF and comparing it to the theoretical water content of Form B, determined by KF or by XRD.

The present invention further provides ezetimibe having a plate morphology.

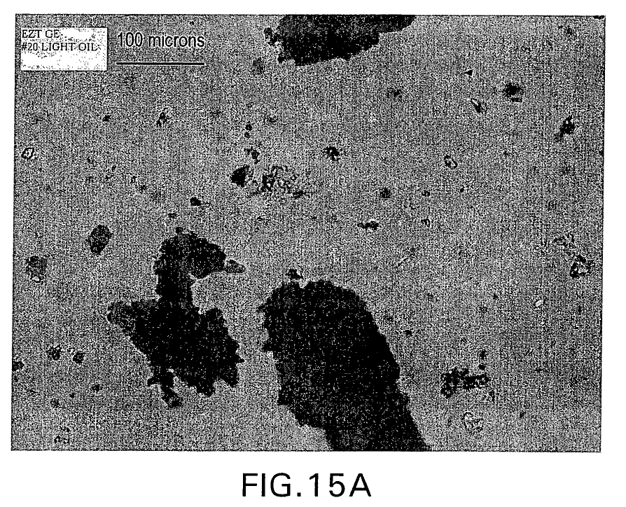

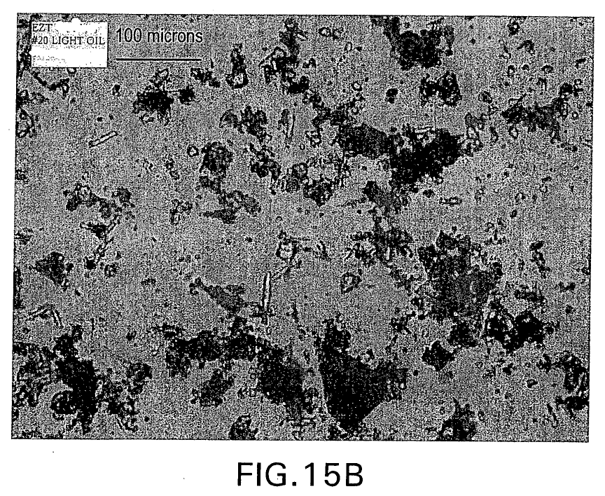

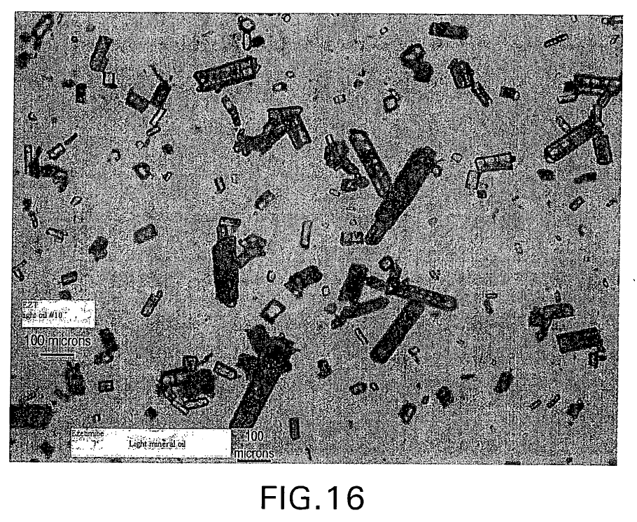

Ezetimibe having a plate morphology contains a negligible amount of agglomerates as compared to the needle-shaped ezetimibe which has many agglomerates, as depicted in FIGS. 15a and 15b. It is known that needle shaped crystals with agglomerates cause processability problems, for example, sticking due to greater static electricity, and being less compact as compared to plate shaped particles. Therefore, the morphology of the crystal form is a determining factor in pharmaceutical formulations. Ezetimibe having a plate-shaped morphology, as depicted in FIG. 16, is therefore more preferred for pharmaceutical formulations.

Ezetimibe having a plate morphology can be prepared by combining crude ezetimibe with isopropanol to obtain a solution. The solution is then heated to a temperature of about 45° C. to about 55° C. under stirring and further maintained for about 30 minutes to about one hour to ensure complete dissolution, followed by addition of water, to obtain a suspension. The suspension is then cooled to a temperature of about 20° C. to about 10° C. to induce precipitation, followed by the recovery of the precipitate. Crude ezetimibe may be obtained commercially.

Preferably, the solution is filtered through a mechanical filter to dispose of particles other than ezetimibe. More preferably, the filtration is done while maintaining the temperature of the solution and the filtrate, for example, by hot filtration.

Preferably, water is added in a drop-wise manner, more preferably, over a period of about 20 minutes to about an hour.

Preferably, the suspension is maintained at a temperature of about 45° C. to about 55° C., more preferably, under stirring, for about 15 minutes to about a half an hour.

Preferably, the suspension is cooled over a period of about two hours followed by maintaining, more preferably, under stirring, for about two hours, to induce precipitation.

The precipitate may be recovered by filtration using a centrifuge, followed by washing with water and drying in a vacuum oven at a temperature of about 55° C. to about 65° C. for about 5 to about 20 hours.

Morphology of the recovered precipitate may be determined by microscope observation.

The present invention provides a pharmaceutical composition containing ezetimibe prepared according to the processes of the present invention, and at least one pharmaceutically acceptable excipient.

The present invention also provides a pharmaceutical composition containing at least one of micronized ezetimibe Form A, micronized ezetimibe Form B, and ezetimibe having a plate morphology, and at least one pharmaceutically acceptable excipient.

The present invention further provides a process for preparing a stable pharmaceutical formulation by combining ezetimibe prepared according to the processes of the present invention with a pharmaceutically acceptable carrier.

The present invention further provides a process for preparing a stable pharmaceutical formulation by combining at least one of micronized ezetimibe Form A, micronized ezetimibe Form B, and ezetimibe having a plate morphology with at least one pharmaceutically acceptable excipient.

The ezetimibe forms described herein can be formulated into a variety of compositions for administration to humans and animals for treating diseases through the reduction of cholesterol.

Methods of administration of a pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

Pharmaceutical compositions of the present invention can optionally be mixed with other forms of ezetimibe and/or other active ingredients such as HMG-CoA reductase inhibitors. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol and talc.

Carriers for use in the pharmaceutical compositions may include, but are not limited to, lactose, white sugar, sodium chloride, glucose, urea, starch, calcium carbonate, kaolin, crystalline cellulose, silicic acid, and the like.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

Disintegrants can increase dissolution. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like.

Absorption accelerators may include, but are not limited to, quaternary ammonium base, sodium laurylsulfate, and the like.

Wetting agents may include, but are not limited to, glycerin, starch, and the like. Adsorbing agents used include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the ezetimibe forms described herein and any other solid ingredients are dissolved or suspended in a liquid carrier, such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste. Preservatives and chelating agents such as alcohol, sodium benzoate, butylated hydroxy toluene, butylated hydroxyanisole and ethylenediamine tetraacetic acid can be added at safe levels to improve storage stability.

A liquid composition according to the present invention can also contain a buffer such as guconic acid, lactic acid, citric acid or acetic acid, sodium guconate, sodium lactate, sodium citrate or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added. If necessary, coloring agents, preservatives, perfumes, seasoning agents, sweetening agents, and other medicines may also be added to the desired preparations during the treatment of schizophrenia.

The amount of ezetimibe or pharmaceutically acceptable salt thereof contained in a pharmaceutical composition for reducing cholesterol according to the present invention is not specifically restricted; however, the dose should be sufficient to treat, ameliorate, or reduce the condition. For example, ezetimibe may be present in an amount of about 1% to about 70%.

The dosage of a pharmaceutical composition for reducing cholesterol according to the present invention will depend on the method of use, the age, sex, weight and condition of the patient. Typically, about 1 mg to 200 mg of ezetimibe may be contained in an administration unit form, preferably a 10 mg tablet.

Having described the invention with reference to certain preferred embodiments, other embodiments will become apparent to one skilled in the art from consideration of the specification. The invention is further defined by reference to the following examples describing in detail the analysis of the crystals and processes for making the crystals of the invention. It will be apparent to those skilled in the art that many modifications, both to materials and methods, may be practiced without departing from the scope of the invention.

### EXAMPLES

In the following examples, the forms of ezetimibe were identified using Scintag X-ray powder diffractometer model X'TRA, Cu-tube solid state detector. The sample holder was a round standard aluminum sample holder with rough zero background quartz plate with a cavity of 25 (diameter) X 0.5 mm (depth). The scanning parameters were range: 2-40 degrees two-theta; scan mode: continuous scan; step size: 0.05 deg.; and a rate of 3 deg/min.

To determine the “loss on dry” (LOD) by thermal gravimetric analysis (TGA), the sample was heated from about 25° C. to about 200° C. at a heating rate of about 10° C. per minute, while purging with nitrogen gas at a flow rate of 40 ml/min.

Quantification of Form B can be done by methods known in the art such as XRD analysis. The ratio between the areas of a peak of Form B and a peak of Form A may be computed. Alternatively, the ratio between the area of a peak of Form B and the total area of the diffractogram may be computed.

Quantification of Form B can also be done by measuring the water content. Theoretical and measured percentages of Form A and Form B are summarized in Table 1.

TABLE 1
Form A and Form B content based on the measured water
content of Form A and Form B mixture

| | | | Measured water |
|---|---|---|---|
| Form A | Form B | Theoretical water | content of Form A |
| content | content | content of Form B | and Form B mixture |
| (%) | (%) | (wt %) | (wt %) |
| 0 | 100 | 4.2 | 4.2 |
| 10 | 90 | 3.8 | 3.9 |
| 20 | 80 | 3.4 | 3.4 |
| 40 | 60 | 2.6 | 2.6 |
| 60 | 40 | 1.8 | 1.9 |
| 80 | 20 | 1.1 | 1.1 |
| 100 | 0 | — | 0.2 |

TABLE 2
Representative XRD peaks of Form A and Form B
(±0.2 degrees two-theta).

| Form A | Form B |
|---|---|
| 8.2 | 8.0 |
| 13.5 | 21.9 |
| 16.3 | 24.6 |
| 25.6 | 26.5 |
| | 15.9 |

Particle Size Determination:
Ezetimibe Samples PSD Measurement by Malvern Laser Diffraction

A Malvern Laser Diffraction instrument was used to characterize the particle size distribution of ezetimibe. A Mastersizer S model equipped with a small cell dispersion unit MS1 with a digital dispersion unit controller was used. The measurement was done using range lens 300RF (working range 0.05-900 mcm), beam length: 2.40 mm and presentation 3NHE. In this case, a solution of dioctyl sulfosuccinate sodium salt in n-hexane was used as a dilution medium. The measurement was started after 1 minute of recirculation after suspension addition into measurement cell at speed rate 2000±10 rpm.

The suspension was prepared of 0.1 g sample in solution 0.065% dioctyl sulfosuccinate sodium salt in n-hexane by vortex for 10 seconds and by sonication for 30 seconds. According to the accepted rules of Good Manufacture Procedures, the sample of ezetimibe is preferably measured after a successful blank measurement (% obscuration NMT 0.1% is performed.

### Example 1

### Particle Size Measurement of Ezetimibe Form A

Malvern, PSD, results of prime particles with sonication were as follows:

d(0.1): 0.39 microns, d (0.5): 3.18-3.33 microns, d (0.9): 7.95-8.05 microns.

### Example 2

### Preparation of Ezetimibe Forms by Single Solvent Crystallization

4 g of ezetimibe was dissolved in a solvent. The choice and volume of the solvent is shown in Table 3.

The mixture was stirred and heated to reflux. The reflux temperature is shown in Table 1. The solution was cooled under stirring to room temperature and then left at 4° C. for 16 h. The resulting precipitate was filtered using a Buchner funnel and sucked dry for 30 min on the funnel. The resulting sample, the “wet sample,” was studied by XRD. The wet sample was then dried under reduced pressure for 16 h. The dry sample was studied by XRD. The crystalline forms obtained from the wet and dry samples are shown in Table 3.

TABLE 3

| | Temp | Vol. | | |
|---|---|---|---|---|
| Solvent | (° C.) | (ml) | Dry Result | Wet Result |
| methyl isobutyl | 116.5 | 10 | Form A | Form B |
| ketone | | | | |
| n-butanol | 118 | 14 | Form A | Forms A + B |
| n-propanol | 97 | 14 | Form A | Form A + ˜20% B* |
| Butyl Acetate | 124-126 | 14 | Form A | Form A + B |
| isoamyl alcohol | 130 | 10 | Form A | Form A + 8.3 + |
| | | | | 8.8 + 9.2 + 12.1 + |
| | | | | 24.6 + 26.5 |
| Bromobenzene | 156 | 10 | Form A | Form A + B |
| Chlorobenzene | 100-102 | 10 | Form A | Form A + ˜10% B* |
| Nitromethane | 101 | 10 | Form A | Form B + ˜30% A* |
| Dibromomethane | 96-98 | 10 | Form A | Form B + ˜15% A* |
| Dibromobutane | 194 | 10 | Form A | Form A + ˜10% B* |
| Ethylene glycol | 196 | 10 | Essentially | Essentially |
| | | | Amorphous | Amorphous |
| Xylene | 139 | 10 | Form A | Form A + ˜10% B* |
| Toluene | 110.6 | 80 | Form A | Form A + ˜30% B* |
| DCM | 40 | 250 | Form A | Form B |
| Chloroform | 61 | 175 | Form A | Form B |
| ACN | 81-82 | 10 | Form A | Form B + ˜30% A* |
| Ethyl acetate | 77 | 10 | Form A | Form B |
| isobutanol | 108 | 10 | Form A | Form A + ˜30% B* |
| 2-butanol | 100 | 10 | Form A | Essentially |
| | | | | Amorphous |

*Estimated from XRD.

### Example 3

### Preparation of Ezetimibe Forms by Solvent-Anti-Solvent Crystallization

4 g ezetimibe was dissolved under stirring in a solvent at reflux conditions. The choice and volume of solvent and the reaction temperature for each experiment is shown in Table 4. An anti-solvent was added until precipitation began. The suspension obtained was cooled under stirring to room temperature and then left overnight at 4° C.

The resulting precipitate was filtered using a funnel and filter paper and dried for 30 min on the funnel/paper. The resulting sample, the “wet sample,” was studied by XRD.

The wet sample was dried under reduced pressure at 70° C. for 16 h and studied by XRD. The crystalline forms obtained from the wet and dry samples are shown in Table 4.

TABLE 4

| | | | | Anti- | | | |
|---|---|---|---|---|---|---|---|
| | | Solvent | | Solvent | | | |
| | b.p. | Vol. | Anti- | Volume | Temp | | |
| Solvent | (° C.) | (ml) | Solvent | (ml) | (° C.) | Dry Result | Wet Result |
| THF | 66 | 10 ml | water | 20 ml | reflux | Form A | Form B |
| Ether | 34.6 | 250 ml | water | 50 ml | RT | Form A | Form B |
| t-Butyl- | 53 | 70 ml | water | 40 ml | RT | Form A | Form B |
| methylether | | | | | | | |
| Acetone | 56 | 10 ml | water | 15 ml | reflux | Form A | Form B |
| Ethanol | 78 | 10 ml | water | 5 ml | reflux | Form A | Form B |
| Acetone | 56 | 10 ml | cyclohexane | 48 ml | RT | Form A | Form B |
| 2-Propanol | 82 | 10 ml | cyclohexane | 40 ml | reflux | Form A | Form B |
| Ethanol | 78 | 10 ml | cyclohexane | 70 ml | reflux | Form A | Form B |
| THF | 66 | 10 ml | cyclohexane | 40 ml | reflux | Form A | Form B |
| 1,3 | 74-75 | 10 ml | cyclohexane | 22 ml | reflux | Form A | Form B |
| dioxolane | | | | | | | |
| 1,3 | 74-75 | 10 ml | water | 6 ml | reflux | Form A | Form B |
| dioxolane | | | | | | | |
| 1,4 Dioxane | 100-102 | 10 ml | cyclohexane | 23 ml | reflux | Form A | Form B |
| MEK | 80 | 10 ml | cyclohexane | 40 ml | reflux | Form A | Form B |
| MEK | 80 | 10 ml | water | 20 ml | reflux | Form A | Form B |
| Propylene | 189 | 10 ml | cyclohexane | 50 ml | reflux | Essentially | Essentially |
| glycol | | | | | | amorph | amorph |
| Propylene | 189 | 10 ml | water | 6 ml | reflux | Form A | Form B |
| glycol | | | | | | | |
| 1,4 Dioxane | 100-102 | 10 ml | water | 9 ml | reflux | Form A | Form B |
| DMF | 153 | 10 ml | water | 10 ml | reflux | Form A | Form B |

### Example 4

### Preparation of Ezetimibe Form B by Slurry

Ezetimibe Form A was stirred in soft water (25 ml) for 4 h at room temperature (heavy slurry). The mixture was filtered and washed with water (20 ml). The wet sample, after washing, was Form B. 97% yield.

### Example 5

### Preparation of Ezetimibe Form B by Slurry

Ezetimibe Form A was stirred in absolute Methanol (2 ml) for 4 h at room temperature. The mixture was filtered. The wet sample, after filtration, was Form B. 15% yield.

### Example 6

### Preparation of Ezetimibe Form B by Slurry

Ezetimibe Form A was stirred in absolute ethanol (4 ml) for 4 h at room temperature. The mixture was filtered. The wet sample, after filtration, contained Form A and Form B (20-30% by weight). 69% yield.

### Example 7

### Preparation of Ezetimibe Form B by Slurry

Ezetimibe Form A was stirred in absolute ethanol (9 ml) for 5 h at room temperature. The mixture was filtered and washed with the filtrate solution. The wet sample, after washing, was Form B.

### Example 8

### Stability of Form B in Water Slurry

Ezetimibe Form B was stirred in soft water (25 ml) for 4 h at room temperature (heavy slurry). The mixture was filtered and washed with water (20 ml). The wet sample, after washing, was Form B. The solid was dried under vacuum at 45° C. for 18 hrs. The dry sample contained Form B (20-30% by weight) and Form A. 95% yield.

### Example 9

### Preparation of Form B Ezetimibe having a Water Content of 4%

3.2 g of ezetimibe was dissolved in 19 ml of ethanol at room temperature. Water (16 ml) was then added, and the precipitated product was isolated by vacuum filtration and dried in vacuum oven at 45° C. for 1 hr. The product was then kept in a refrigerator.

The product was recrystallized by first dissolving it in 19 ml of ethanol at 70° C. Water (16 ml) was then added, and the precipitated product was isolated by vacuum filtration and washed with 5 ml of water to obtain wet ezetimibe Form B.

The sample was dried in the hood for two days, and the crystal form obtained was identified as Form B containing 4.1% water by weight based on Karl Fischer analysis.

### Example 10

### Preparation of Amorphous Ezetimibe

0.6 g ezetimibe was dissolved in 20 ml acetone at room temperature. The solution was fed for 5 min into pre-heated (60° C.) 1 L reactor under vacuum (˜20 mmHg) to obtain Amorphous ezetimibe.

### Example 11

### Transformation of Ezetimibe Crystal Forms By Heating

500 mg samples were placed in an open glass bottle and inserted into an atmospheric oven at various temperatures and for about 2 hours. After two hours, each sample was removed and analyzed by XRD. The results are summarized below in Table 5.

TABLE 5

| | HEAT TRANSFORMATION | | |
|---|---|---|---|
| | TEMP | TIME | |
| FORM | (° C.) | (h) | RESULT |
| Form A | 60 | 2 | Form A |
| | 90 | 2 | Form A |
| | 110 | 2 | Form A |
| Form B | 45 | 18 | Form A |
| | 60 | 2 | Form A |
| | 90 | 2 | Form A |
| | 110 | 2 | Form A |
| Amorphous | 60 | 2 | Amorphous Form |
| Form | 90 | 2 | Form A |

*The drying was done in a vacuum oven

### Example 12

### Transformation of Ezetimibe Form A Under Different Relative Humidities

500 mg samples were placed in an open glass bottle at a temperature of 25° C. to 30° C., under various humidity for a sufficient time (1-14 days) for each sample to equilibrate. At the end of each time period, each sample was removed and analyzed by XRD and by KF. The results are summarized below in Tables 6-7 for starting Forms A and B, respectively.

TABLE 6
Form A

| Relative | Water content by | | |
|---|---|---|---|
| humidity (%) | KF (wt %) | Time (days) | Crystal form |
| 0 | 0.2 | 12 | Form A |
| 20 | 0.2 | 7 | Form A + ˜5% B* |
| 40 | 0.3 | 12 | Form A + ˜5-10% B* |
| 80 | 4.5 | 12 | Form B |
| 100 | 4.3 | 1 | Form B |

*This concentration is estimation. Form A transforms to Form B at about 20-100% RH.

TABLE 7
Form B

| Relative | Water content by | | |
|---|---|---|---|
| humidity (%) | KF (wt %) | Time (days) | Crystal form |
| 0 | 0.1 | 14 | Form A |
| 20 | 0.2 | 7 | Form A + ˜5% B* |
| 40 | 4.0 | 14 | Form B |
| 80 | 4.3 | 14 | Form B |
| 100 | 4.4 | 14 | Form B |

*Form B transforms to anhydrous Form A at 0% RH.

### Comparative Example 13

### Preparation of Form A Ezetimibe according to WO 2004/99132

Ezetimibe was dissolved in ethyl acetate and hexane was added, as described in WO 2004/99132. The mixture of stirred for about 2 hours, and the crystals obtained were collected by filtration to yield a wet product. The wet product was then dried. The wet and dry samples were identified as Form A.

### Example 14

### Micronization of Form B Ezetimibe

Form B was milled by a 50 mm micronizer for about 20 to 30 minutes. The feed air rate was 6.0 bar and the grinding air was 5.0 bar. The sample was analyzed before and after micronization by XRD. During micronization 65% (estimated by XRD and by KF) of Form B converted to Form A. The estimated water content is 1-2% by weight. Micronization of Form A on the other hand results in Form A.

1 g mixture of micronized Form A and B was stored at 30° C. under 100% RH for 8 days. The mixture of Form A and Form B converted to 100% Form B after storage. Micronized Form B contained 4.1% water by weight based on Karl Fischer analysis. The micronized sample was analyzed before and after storage by XRD. The material was dispersed in light mineral oil before measurement for microscope observation. Form B was in the shape of needles and plates.

Specific surface area was measured using the following parameters:

Instrument: Coulter SA3100

Degassing: without

Sensitivity: High

Calculation: BET

Type: Multipoint

Points: 10

Sample cell: 9 cm³

Table 8 illustrates the maximum particle size and specific surface area of ezetimibe Form B before and after micronization. The maximum particle size was determined by microscope observation.

TABLE 8
Maximum particle size and specific surface area
of ezetimibe Form B.

| | SSA (m²/g) | Maximum particle size (μm) |
|---|---|---|
| Before micronization | 3.4 | 400 |
| After micronization | 6.8 | 20 |

### Example 15

### Effect of Pressing on Form A and Form B

Forms A and B were studied under pressure. The forms were pressed for 1 minute under a pressure of approximately 1300 psi. The results are summarized in Table 9.

TABLE 9
Effect of pressing on ezetimibe Form A and Form B

| Sample before pressing | Sample after pressing |
|---|---|
| Form B | Form B |
| Form A | Form A + ˜5% B |

### Example 16

### Preparation of Ezetimibe having a Plate Morphology

Ezetimibe crude (7 kg) was dissolved in iso-propanol (35 L). The solution was heated to 50° C. under stirring and is further maintained for at least 30 minutes to ensure complete dissolution, followed by filtration through a mechanical filter and washing with IPA (7 L). The filtrate was reheated to 50° C., followed by adding process water (17.5 L) over 20 min, to obtain a suspension. The suspension was stirred at 50° C. for at least 15 min, followed by cooling, under stirring, to 10-20° C. over 2 h and then, stirring at 10-20° C. for additional 2 h. The suspension was filtered using a centrifuge and washed with of process water (7 L), followed by drying under vacuum at 55-65° C. for 5-20 hr, to give the ezetimibe, having a plate morphology.

What is claimed is:

1. A process for obtaining ezetimibe Form B comprising:
(a) combining ezetimibe with a solvent including at least one solvent selected from the group consisting of methyl isobutyl ketone, dichloromethane, chloroform, and ethylacetate to obtain a mixture;
(b) heating the mixture of step (a) at a temperature sufficient to obtain a solution;
(c) precipitating the ezetimibe from the solution of step (b); and
(d) optionally recovering the precipitate.

2. A process for obtaining a mixture of ezetimibe Form A and Form B comprising:
(a) combining ezetimibe with a solvent including at least one solvent selected from the group consisting of n-butanol, n-propanol, butylacetate, bromobenzene, chlorobenzene, dibromomethane, xylene, toluene, acetonitrile, nitromethane, and isobutanol to obtain a mixture;
(b) heating the mixture of step (a) at a temperature sufficient to obtain a solution;
(c) precipitating the ezetimibe from the solution of step (b); and
(d) optionally recovering the precipitate.

3. A process for obtaining ezetimibe Form A comprising:
(a) combining ezetimibe with a solvent including isoamyl alcohol to obtain a mixture;
(b) heating the mixture of step (a) at a temperature sufficient to obtain a solution;
(c) precipitating the ezetimibe from the solution of step (b); and
(d) optionally recovering the precipitate.

4. A process for obtaining amorphous ezetimibe comprising:
(a) combining ezetimibe with a solvent including at least one solvent selected from the group consisting of ethylene glycol and 2-butanol to obtain a mixture;
(b) heating the mixture of step (a) at a temperature sufficient to obtain a solution;
(c) precipitating the ezetimibe from the solution of step (b); and
(d) optionally recovering the precipitate.

5. A process for obtaining ezetimibe Form B comprising:
(a) combining ezetimibe with a solvent including at least one solvent selected from the group consisting of an ether, a ketone, an amide, methanol, ethanol, 2-propanol, and propylene glycol to obtain a mixture;
(b) heating the mixture of step (a) at a temperature sufficient to obtain a solution;
(c) combining the solution of step (b) with a solvent including at least one anti-solvent selected from the group consisting of water and a cyclic or linear C₅₋₆ aliphatic hydrocarbon to obtain a suspension;
(d) precipitating the ezetimibe from the suspension of step (c); and
(e) optionally recovering the precipitate.

6. The process of claim 4, wherein the ether is tetrahydrofuran, diethylether, t-butyl-methylether, 1,3-dioxalane or 1,4-dioxane.

7. The process of claim 4, wherein the ketone is acetone or methylethyl ketone.

8. The process of claim 4, wherein the amide is N,N-dimethylformamide.

9. The process of claim 4, wherein the C₅₋₆ aliphatic hydrocarbon is cyclohexane.

10. A process for obtaining amorphous ezetimibe comprising:
(a) combining ezetimibe with a solvent including propylene glycol to obtain a mixture;
(b) heating the mixture of step (a) at a temperature sufficient to obtain a solution;
(c) combining the solution of step (b) with a solvent including at least one anti-solvent selected from the group consisting of water and a cyclic or linear C₅₋₆ aliphatic hydrocarbon to obtain a suspension;
(d) precipitating the ezetimibe from the suspension of step (c); and
(e) optionally recovering the precipitate.

11. A process for preparing ezetimibe Form B comprising slurrying ezetimibe Form A in a solvent including at least one solvent selected from the group consisting of water and a C₁₋₄ alcohol.

12. The process of claim 11, wherein the C₁₋₄ alcohol ethanol or methanol.

13. The process of claim 11, wherein Form A is slurried at a temperature of about 15° C. to about 30° C.

14. The process of claim 13, wherein Form A is slurried for about 3 to about 8 hours.

15. A process for preparing ezetimibe Form B comprising:
(a) combining ezetimibe with a solvent including a C₁₄ alcohol to obtain a solution;
(b) combining the solution of step (a) with water to obtain a precipitate;
(c) recovering the precipitate; and
(d) recrystallizing the precipitate of step (c).

16. The process of claim 15, wherein the C ₁₄ alcohol is ethanol.

17. The process of claim 15, wherein the recrystallization of step (d) comprises:
(e) combining the precipitate of step (c) with a C₁₋₄ alcohol to obtain a solution; and
(f) combining the solution of step (e) with water to obtain ezetimibe Form B.

18. The process of claim 15, wherein Form B contains about 3% to about 5% water by weight as determined by KF analysis.

19. The process of claim 18, wherein Form B contains about 4.1% of water by weight as determined by KF analysis.

20. A process for preparing ezetimibe Form A comprising maintaining ezetimibe Form B or amorphous ezetimibe at a temperature of about 40° C. to about 110° C. for about 2 hours to about 18 hours.

21. A process for preparing ezetimibe Form B comprising exposing ezetimibe Form A to a relative humidity of about 40% to about 100% for about 1 day to about 14 days at a temperature of about 25° C. to about 30° C.

22. The process of claim 21, wherein the relative humidity is about 100% and Form A is converted to Form B in about 1 day.

23. A process for preparing ezetimibe Form A comprising exposing ezetimibe Form B to a relative humidity of about 0% to about 20% for about 7 days to about 14 days at a temperature of about 25° C. to about 30° C.

24. The process of claim 23, wherein Form B is converted to Form A in less than about 3 days.

25. The process of claim 23, wherein the Form A obtained is in an amount greater than any other single ezetimibe polymorphic form by weight.

26. The process of claim 25, wherein about 90% to about 95% of Form A by weight is obtained.

27. The process of claim 23, wherein about 100% of Form A by weight of the ezetimibe is obtained.

28. A process for preparing Form A comprising micronizing Form B.

29. The process of claim 28, wherein the micronization is done by milling Form B.

30. The process of claim 29, wherein Form B is milled at a feed air rate of about 6 bar and a grinding air pressure of about 5 bar for about 20 to about 30 minutes.

31. The process of claim 29, wherein Form B is milled for a maximum of about 30 minutes.

32. The process of claim 31, wherein the Form A obtained contains about 35% of Form B and about 1% to about 2% of water by weight.

33. The process of claim 32, wherein complete transformation of Form B to Form A occurs, as determined by XRD or by KF.

34. A process for preparing Form B by exposing a mixture of micronized Form A and micronized Form B to a relative humidity of about 40% to about 100% at a temperature of 25° C. about 30° C. for about 7 to about 14 days.

35. The process of claim 34, wherein the obtained Form B contains about 3% to about 5% of water by weight.

36. The process of claim 35, wherein the obtained Form B contains about 4.1% of water by weight.

37. The process of claim 34, wherein the mixture of micronized Form A and micronized Form B is obtained by micronizing Form B.

38. The process of claim 34, wherein the mixture of micronized Form A and micronized Form B converts to Form B in less than about 7 days.

39. Ezetimibe prepared according to the process of claims 1-38.

40. Micronized ezetimibe Form A.

41. The micronized ezetimibe Form A of claim 40 wherein at least 99% of micronized ezetimibe has a particle size of less than about 30 microns 28.

42. The micronized ezetimibe Form A of claim 40 wherein at least 99% of micronized ezetimibe has a particle size of less than about 20 microns.

43. The micronized ezetimibe Form A of claim 40 wherein at least 99% of micronized ezetimibe has a particle size of less than about 10 microns.

44. Micronized ezetimibe Form B.

45. The micronized ezetimibe Form B of claim 44 wherein at least 99% of micronized ezetimibe has a particle size of less than about 30 microns.

46. The micronized ezetimibe Form B of claim 44 wherein at least 99% of micronized ezetimibe has a particle size of less than about 20 microns.

47. The micronized ezetimibe Form B of claim 44 wherein at least 99% of micronized ezetimibe has a particle size of less than about 10 microns.

48. Ezetimibe having a plate morphology.

49. A pharmaceutical composition comprising the ezetimibe of any of claims 39 to 48, and at least one pharmaceutically acceptable excipient.

50. A process for preparing a stable pharmaceutical formulation comprising combining the ezetimibe of any of claims 39 to 48 and at least one pharmaceutically acceptable excipient.
